(19) United States
(12) Patent Application Publication
(10) Pub. No.: US 2015/0265336 A1
(43) Pub. Date: Sep. 24, 2015

(54) MICRO-MECHANICAL DEVICE AND METHOD FOR OBSTRUCTIVE SLEEP APNEA TREATMENT

(71) Applicant: MICROFABRICA INC., Van Nuys, CA (US)

(72) Inventors: Gregory P. Schmitz, Los Gatos, CA (US); Gregory B. Arcenio, Redwood City, CA (US); Eric C. Miller, Los Gatos, CA (US)

(21) Appl. No.: 14/440,088

(22) PCT Filed: Nov. 20, 2013
(86) PCT No.: PCT/US2013/070909

§ 371 Date: May 1, 2015

Related U.S. Application Data
(60) Provisional application No. 61/728,443, filed on Nov. 20, 2012.

(51) Int. Cl. A61B 18/14; A61B 18/20; A61B 1/00; A61B 18/18

(52) U.S. Cl. CPC A61B 18/1482; A61B 18/1815; A61B 18/20; A61B 1/00087; A61B 2018/1861; A61B 2018/202; A61B 2018/00327

(57) ABSTRACT

A method for removing a volume of tissue from a tongue in a patient to treat sleep apnea may involve cutting tissue from the tongue using a tissue cutting device having a shaft and at least one moveable cutting member attached to the shaft at a distal end of the tissue cutting device and moving the cut tissue through a channel of the shaft in a direction from the distal end of the tissue cutting device toward a proximal end of the device. A device for removing a volume of tissue from a tongue in a patient to treat sleep apnea may include a shaft, at least one moveable cutting member disposed at a distal end of a distal tip of the shaft, a handle coupled with a proximal portion of the shaft, and an actuator.

### BACKGROUND

1. Field of the Invention

The field of the present application pertains to medical devices. More specifically, the present application is related to methods and systems for treating obstructive sleep apnea, using a micro-mechanical debrider device.

2. Description of the Related Art

Obstructive sleep apnea is a condition in which the flow of air pauses or decreases during breathing while asleep, because the airway has become narrowed, blocked, or floppy. Obstructive sleep apnea causes disturbed sleep, which results in many different side effects, such as daytime sleepiness, irritability, headaches and difficulty concentrating. The condition is becoming increasingly more common, with estimates of as high as 1 in 5 American adults suffering from the disorder. On the whole, obstructive sleep apnea amounts to a large economic expense to society, in terms of medical care as well as lost productivity in the workplace.

One cause of obstructive sleep apnea is a large tongue. The large tongue crowds the mouth and pushes back toward the throat, blocking the airway. The surgical procedure for treating this condition is called partial glossectomy or tongue base reduction and involves removing a portion of the back part (or “base”) of the tongue. This results in less tissue bulk to fall backwards and obstruct breathing. Thus, the airway in the back of the throat is opened, allowing for more comfortable and uninterrupted sleeping at night.

There are two basic types of procedures for performing a tongue base reduction for obstructive sleep apnea—one using RF cautery and one using traditional surgical resection.

For RF cautery, there are two methods employed. Both RF cautery methods rely on destroying the muscle tissue at the back of tongue just below the top surface. For the first RF cautery method, an incision is made at the back of the tongue. The tip of the cautery device is inserted into the incision as shown in FIG. 1. The RF probe used for the first method is typically referred to as a coblation probe. The second method using RF cautery relies on piercing the tongue with a dual-needle-tip RF bipolar probe as shown in FIG. 2. FIGS. 3 and 4 show the insertion of the sharp piercing type bipolar probe and methods of treating the area of the tongue. There are several variables that determine how the treatment is administered for this procedure: time, energy level, and the variation of the patient's tissue impedance. The efficacy of the procedure is not exactly known, but follow-up procedures are often needed to complete the reduction.

The other treatment modality for an enlarged tongue, traditional surgical resection, is done by removing a large portions of the tongue with a scapel and cauterizing the resulting wound. FIG. 5 illustrates several different types of surgical tongue resections that are used for reducing the size of the tongue.

Although current procedures for tongue base reduction may be effective in some patients, in many cases RF-based procedures may produce little or no reduction in symptoms, and surgical tissue removal typically involves a painful and lengthy recovery (while still not always ameliorating the condition). Therefore, it would be beneficial to have improved apparatus and methods for performing a tongue base tissue reduction procedure.

### BRIEF SUMMARY

Example embodiments described herein have several features, no single one of which is indispensable or solely responsible for their desirable attributes. Without limiting the scope of the claims, some of the advantageous features of some embodiments will now be summarized.

In one aspect, a method for removing a volume of tissue from a tongue in a patient to treat sleep apnea may involve cutting tissue from the tongue using a tissue cutting device having a shaft and at least one moveable cutting member attached to the shaft at a distal end of the tissue cutting device and moving the cut tissue through a channel of the shaft in a direction from the distal end of the tissue cutting device toward a proximal end of the device. In some embodiments, the method may further involve, before cutting the tissue, forming an incision in the tongue, and advancing the distal end of the tissue cutting device through the incision to cut tissue within an inner portion of the tongue. In some embodiments, the incision may be formed using the tissue cutting device. In some embodiments, the incision may be formed in the top of the tongue. Alternatively, the incision may be formed in the bottom of the tongue. In yet another alternative embodiment, the incision may be formed from under the patient's chin through the bottom of the tongue.

In some embodiments, the method may further involve closing the incision using an energy emitting member on the tissue cutting device. For example, the energy emitting member may emit energy such as, but not limited to, radiofrequency, ultrasound, microwave, heat or laser energy. In some embodiments, the moveable cutting member may include at least one moveable blade and at least one stationary blade, and cutting the tissue may involve rotating the rotating blade(s) past the stationary blade(s). In some embodiments, the moveable cutting member may include at least two interdigitated tissue cutters, and cutting the tissue may involve rotating the two interdigitated cutters toward one another.

Some embodiments may use application of suction to help move the cut tissue through the channel. Alternatively or additionally, irrigation fluid may be introduced, via the tissue cutting device, to an area at or near the distal end of the tissue cutting device, and applied suction may be used to move at least some of the fluid proximally through the channel with the cut tissue.

In various embodiments, the components of the tissue removal device may have any of a number of suitable dimensions. For example, in some embodiments, the shaft of the tissue cutting device may have a diameter no greater than about 10 mm, a distal tip having a length of between about 1 mm and about 25 mm, and a bend between a proximal portion of the shaft and the distal tip forming an angle between the proximal portion and the distal tip of between about 1 degree and about 90 degrees.

In some embodiments, the method may further involve visualizing the tissue cutting using a visualization device such as, but not limited to, a straight endoscope, an angled endoscope, a swing prism endoscope, a side viewing endoscope, a flexible endoscope, a CMOS digital camera, an ultrasound device or a scanning single fiber endoscope. In some embodiments, the visualization device may be incorporated into the tissue removal device.

Some embodiments may further include a step of measuring an amount of the removed tissue by filtering the removed tissue from a stream of irrigation fluid. Alternatively, the method may involve measuring an amount of the removed tissue by determining motor torque in the tissue removal device during engagement of the device with the tissue and using at least one of the determined motor torque, a time period of tissue removal or a loading condition to approximate the amount of the removed tissue.

In another aspect, a method for removing a volume of tissue from a tongue in a patient to treat sleep apnea may involve cutting tissue from the tongue using a mechanical, tissue debriding device comprising at least one moveable blade.

In another aspect, a device for removing a volume of tissue from a tongue in a patient to treat sleep apnea may include: a shaft having a proximal portion, a distal tip disposed at an angle relative to the proximal portion, and a channel extending from a distal end of the distal tip through at least part of the proximal portion; at least one moveable cutting member disposed at the distal end of the distal tip and including at least two interdigated blades; a handle coupled with the proximal portion of the shaft; and an actuator coupled with the handle for actuating the at least one moveable cutting member. In some embodiments, the shaft has a diameter no greater than about 10 mm, a distal tip having a length of between about 1 mm and about 25 mm, and a bend between a proximal portion of the shaft and the distal tip forming an angle between the proximal portion and the distal tip of between about 1 degree and about 90 degrees.

In some embodiments, the channel may be a tissue removal channel extending from the distal end of the distal tip to a proximal aperture on the proximal portion through which tissue can be removed from the device. Some embodiments further include a suction port on the proximal portion or the handle for applying suction to the channel. Optionally, embodiments may also include an irrigation port on the proximal portion or the handle for applying irrigation fluid to the channel. In one embodiment, the suction port may be in fluid communication with a suction channel in an inner tube of the device, and wherein the irrigation port is in fluid communication with an irrigation channel comprising a space between an outer surface of the inner tube and an inner surface of the shaft of the device.

In some embodiments, the cutting member may include at least one rotating blade at least one stationary blade positioned relative to the rotating blade such that tissue is cut between the rotating blade and the stationary blade. In some embodiments, the cutting member may include multiple interdigitated cutters that rotate toward one another to shred tissue. Some embodiments may include at least one tubular crown gear for driving the at least one cutting member. In some embodiments, the device may include two tubular crown gears coupled together with at least one intermediate gear disposed between them. For example, the intermediate gear may be disposed at a bend in the shaft located at an intersection of the proximal portion and the distal tip.

In some embodiments, the device may further include an energy transmission member coupled with the distal tip of the shaft for transmitting energy to the tissue. The energy transmitted by the energy transmission member may include, but is not limited to, radiofrequency, ultrasound, microwave, heat and laser energy.

In another aspect, a system for removing a volume of tissue from a tongue in a patient to treat sleep apnea may include a mechanical tissue debrider. The debrider may include: a shaft having a proximal portion, a distal tip disposed at an angle relative to the proximal portion, and a channel extending from a distal end of the distal tip through at least part of the proximal portion; at least one moveable cutting member disposed at the distal end of the distal tip; a handle coupled with the proximal portion of the shaft; and an actuator coupled with the handle for actuating the at least one moveable cutting member. The system may further include an energy transmission member coupled with the distal tip of the shaft for transmitting an energy to the tissue. The energy may include, but is not limited to, radiofrequency, ultrasound, microwave, heat or laser energy.

Some embodiments of the system may further include a suction port on the proximal portion of the shaft or the handle for applying suction to the channel. Some embodiments may further include an irrigation port on the proximal portion of the shaft or the handle for applying irrigation fluid to the channel.

These and other aspects and embodiments of the invention will be described below in further detail, in relation to the attached drawings.

### BRIEF DESCRIPTION OF THE DRAWINGS

FIG. 1 illustrates a prior art method for radiofrequency reduction of a tongue base;

FIG. 2 illustrates a prior art device for radiofrequency reduction of a tongue base;

FIG. 3 illustrates a prior art method for radiofrequency reduction of a tongue base, using a device like the one illustrated in FIG. 2;

FIG. 4 diagrammatically illustrates a tongue, showing areas for a radiofrequency tongue base reduction using the device and method of FIGS. 2 and 3;

FIG. 5 illustrates several versions of a prior art method for surgical reduction of a tongue base;

FIG. 6 is a cross section of a patient's head, showing a method for making an initial incision for performing a tongue base reduction procedure, according to one embodiment;

FIGS. 7A and 7B illustrate two alternative embodiments of positions of initial incisions on a tongue for tongue base reduction, according to alternative embodiments;

FIG. 8 is a cross section of a patient's head, showing a method for performing a tongue base reduction procedure, according to one embodiment;

FIG. 9 is a cross section of a patient's head, showing a method for applying energy to an incision after performing a tongue base reduction procedure, to help close the incision, according to one embodiment;

FIG. 10 is a cross section of a patient's head, showing a method for viewing a tongue base reduction procedure, using a side-facing endoscope, according to one embodiment;

FIG. 11 is a cross section of a patient's head, showing a closed incision in a tongue after a tongue base reduction procedure has been performed, according to one embodiment;

FIG. 12 is a side view of a tongue, illustrating the tongue before and after tongue reduction, according to one embodiment;

FIG. 13 is a cross section of a patient's head, showing a method for performing a tongue base reduction procedure via an entry point through the chin, according to one embodiment;

FIG. 14 is a cross section of a patient's head, showing a method for performing a tongue base reduction procedure via an entry point through the bottom side of the tongue, according to an alternative embodiment;

FIG. 15 is a cross section of a patient's head, showing a method for performing a tongue base reduction procedure via an entry point through the top side of the tongue, according to another alternative embodiment;

FIG. 16 is a side view of a micro-debrider device with interdigitated cutters for performing a tongue reduction procedure for sleep apnea, according to one embodiment;

FIG. 17 is a perspective view of a micro-debrider device with a rotating blade for performing a tongue reduction procedure for sleep apnea, according to an alternative embodiment;

FIG. 18 a perspective view of an angled cutter head of the micro-debrider device of FIG. 16;

FIG. 19 is a perspective view of an angled cutter head of the micro-debrider device of FIG. 17;

FIG. 20 is an exploded view of the distal end of the angled cutter head of FIG. 18;

FIG. 21 is a perspective view of the distal end of the angled cutter head of FIG. 19;

FIG. 22 is a perspective view of the cutting member of the micro-debrider device of FIGS. 16, 18 and 20;

FIG. 23 is a perspective view of the cutting member of the micro-debrider device of FIGS. 17, 19 and 21; and

FIG. 24 is a perspective view of another alternative embodiment of a cutting member for a tissue micro-debrider device.

### DETAILED DESCRIPTION

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

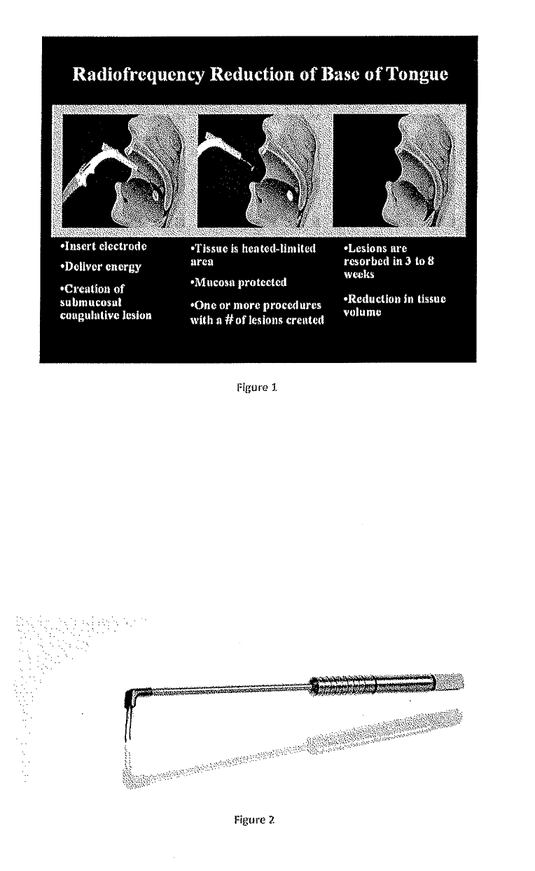

Referring now to FIG. 1, a prior art method is shown for reducing a size of a base of a tongue using a radiofrequency (“RF”) probe. As shown in the panels of FIG. 1, an electrode is first inserted into the base of the tongue, and energy is delivered to create a submucosal lesion. As shown in the second panel, the probe is then removed. Over time, as shown in the third panel, the lesion is resorbed, hopefully causing a reduction in tissue volume at the base of the tongue.

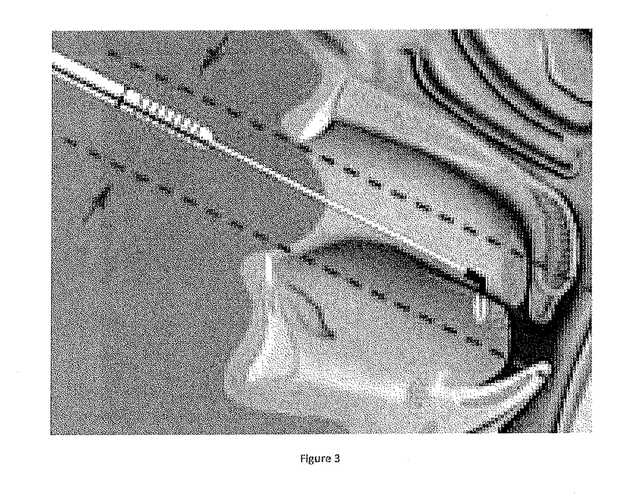

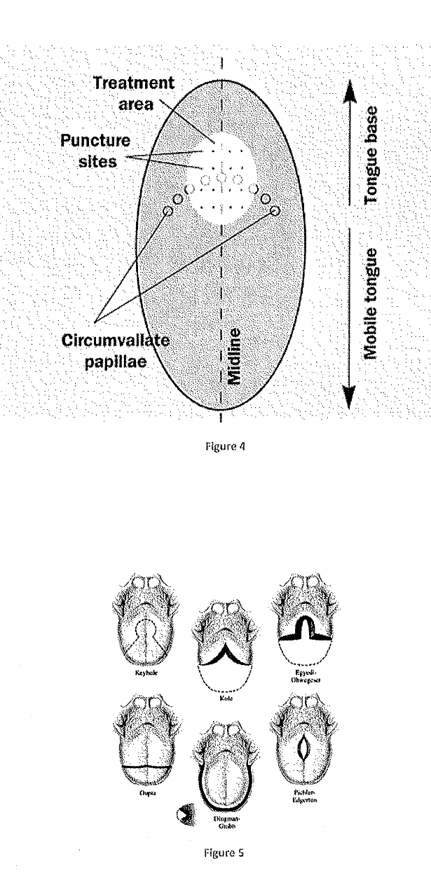

Referring to FIGS. 2-4, another version of an RF probe for reducing the tongue base and a method for using the device are illustrated. This version is called the Radiofrequency Volume Reduction (RaVoR™) probe, manufactured by Sutter Medizintechnik GmbH (www.sutter-med.de). The device includes a bipolar RF electrode for delivering energy to the tongue base in the same way described above. FIG. 4 illustrates a typical pattern for treating a tongue base, using such a probe. One potential disadvantage of RF probes and other, similar energy delivery devices is that their effects on the tongue base and thus on sleep apnea may be minimal. In many cases, it may be necessary to undergo multiple treatments, and even then the effects may be minimal.

Referring to FIG. 5, several different patterns are shown for surgical removal of tissue from the tongue base to treat sleep apnea. The most obvious disadvantage of surgical tissue removal is that it is quite invasive and may lead to a lengthy, painful recovery.

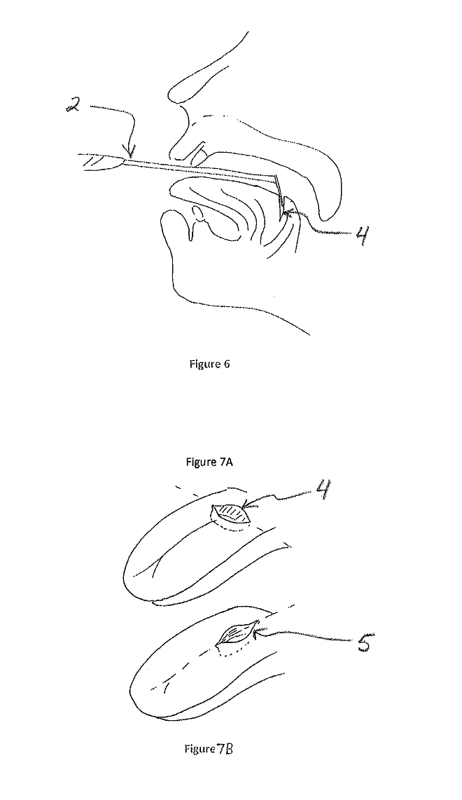

Beginning with FIG. 6, a new method for removing tissue from a tongue base, according to one embodiment, is illustrated. As illustrated in FIG. 6, a first step of the method may involve forming an incision 4 in the tongue, using a cutter 2. In some embodiments, cutter 2 may be the same device used to perform the rest of the tongue reduction procedure. In other embodiments, as in FIG. 6, cutter 2 may be a separate device, which may be provided as part of a tongue reduction system or kit, or which alternatively may be any conventional cutter used by ENT (ear, nose and throat) surgeons.

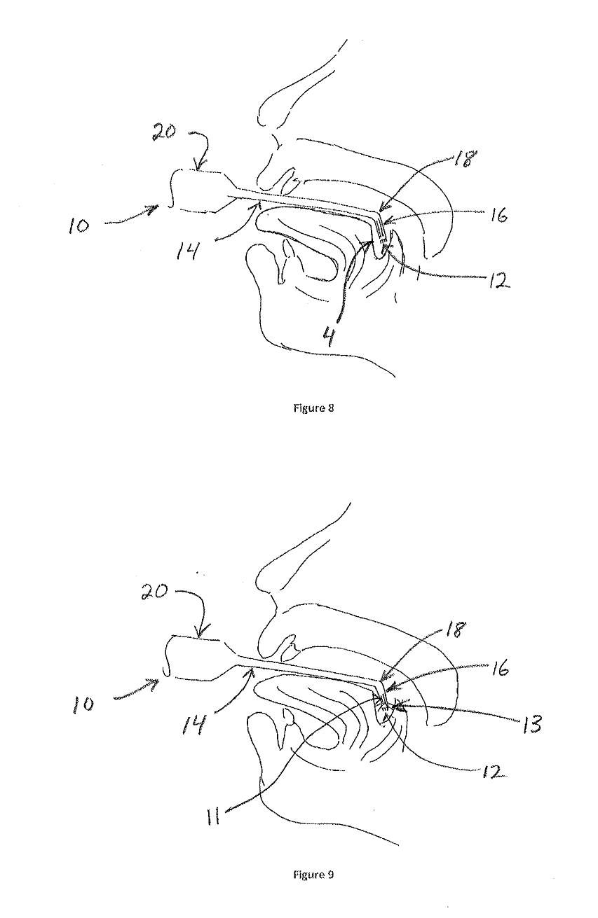

FIGS. 7A and 7B illustrate two different incisions 4 and 5 that may be formed in the tongue base using cutter 2. Of course, other sizes, locations and orientations of incisions are possible for performing the tongue reduction procedure described herein. These examples are simply provided for illustrative purposes.

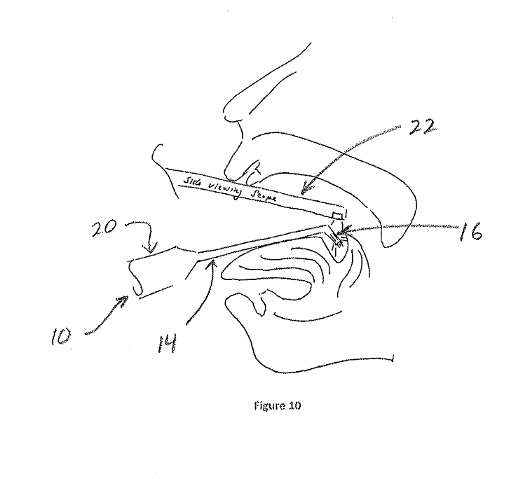

Turning to FIG. 8, once incision 4 has been made in the tongue, a tongue volume reduction micro-debrider device 10 may be inserted into the patient's mouth and advanced so that a cutting member at the distal end 12 of device 10 enters into the incision. Micro-debrider device 10 may generally include a shaft 14, which may include a proximal portion, a distal tip 16, and a bend 18 at the intersection of the proximal portion and the distal tip 16. A handle 20 may be coupled with the proximal portion of the shaft 14 for gripping and actuating device 10. Further details of various embodiments of device 10 are described below in greater detail.

The cutting member (too small to be seen in FIG. 6) of micro-debrider device 10 includes one or more blades or other cutters, at least one of which is moveable. The blades or other cutters are so small that device 10 can remove tissue from the tongue on an individual-fiber level. Cutting and removal of such small pieces of tissue helps minimize trauma and thus pain, bleeding and recovery time for the patient. As tissue is cut by the cutting member, it is pulled into a hollow channel of shaft 14. One mechanism for pulling the tissue into the channel is suction, which may be applied via a suction port on shaft 14 or handle. In some embodiments, irrigation fluid may also be introduced to the tissue removal site via the channel and an irrigation port or via a separate irrigation channel in or on shaft 14. Applying suction to the cut tissue and the irrigation fluid may facilitate removal of the tissue proximally through the shaft.

Referring now to FIG. 9, in some embodiments, micro-debrider device 10 may include one or more energy transmission members 11 in or on shaft 14, for emitting energy 13 to help cauterize bleeding and/or close incision 4. For example, energy transmission members 11 may include, but are not limited to, monopolar RF electrodes, bipolar RF electrodes, or transmitters of heat, ultrasound, microwave or laser energy. Energy 13 may be transmitted for as long as necessary to help stop bleeding and/or close incision 4.

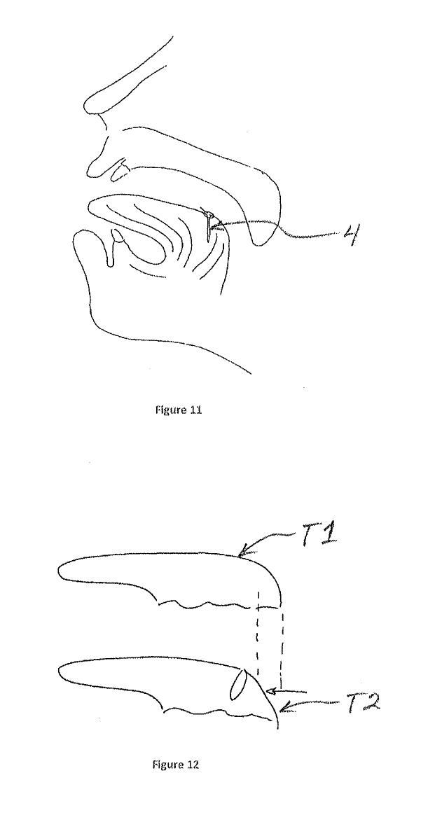

In some embodiments, and with reference now to FIG. 10, a visualization device 22 may be used to visualize some or all of a tongue reduction procedure using micro-debrider device 10. In one embodiment, as shown, visualization device 22 may be a side-viewing endoscope 22. In alternative embodiments, visualization device 22 may be a straight endoscope, an angled endoscope, a swing prism endoscope, a flexible endoscope, a CMOS digital camera, an ultrasound device, a scanning single fiber endo scope, or any other suitable visualization device.

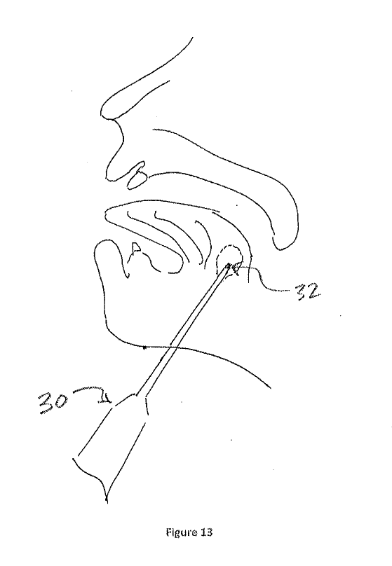

Referring now to FIG. 11, after completion of a tongue reduction procedure as outlined above, incision 4 is closed, and the volume of tissue in the base of the tongue has been reduced. Referring to FIG. 12, when tissue is removed from the base of the tongue in the manner discussed above, the size and shape of the tongue may change, for example, from the configuration shown in T1 (tongue before procedure) to that shown in T2 (tongue after procedure). The reduced volume of the tongue at the base of the tongue may often cause the base of the tongue to shift forward slightly, thus helping eliminate one of the causes of obstructive sleep apnea—i.e., the tongue sliding backward in the patient's airway.

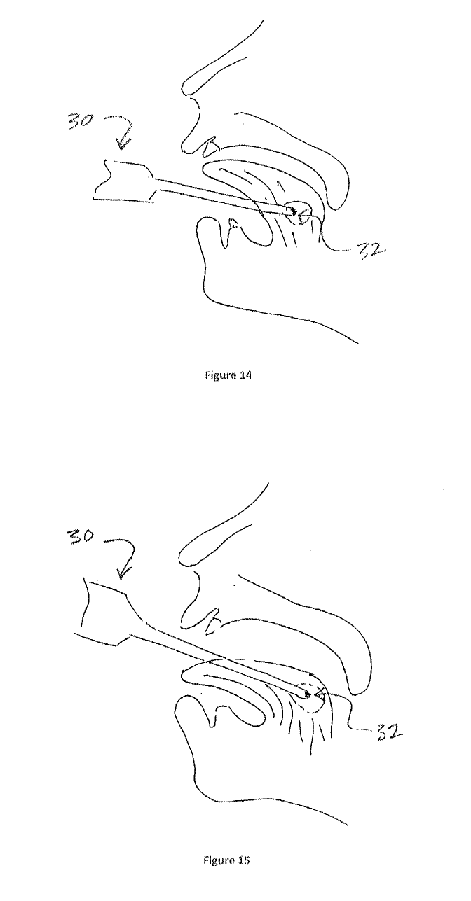

Referring now to FIGS. 13-15, in alternative embodiments, different approaches may be taken for advancing a distal tip 32 of a micro-debrider device 30 into a tongue base for performing tongue base reduction. In some of these alternative embodiments, an alternative micro-debrider device 30, such as the one shown that does not include a bend in its shaft, may be used. As shown in FIG. 13, one alternative approach to the tongue base may be through an incision in the patient's neck, just under the chin. As in FIG. 14, another alternative approach is through an incision in the bottom/underneath surface of the tongue. Finally, as in FIG. 15, another alternative approach is through an incision through the top of the tongue closer to the front of the tongue and advancing in a posterior direction. Other alternative approaches may be used in other alternative embodiments. In any of these embodiments, it may be possible to view the advancement of distal tip 32 via intraoperative fluoroscopy. Alternatively or additionally, some embodiments of device 30 may include depth markers on the shaft for indicating to a user how deeply device 30 has penetrated into tissue.

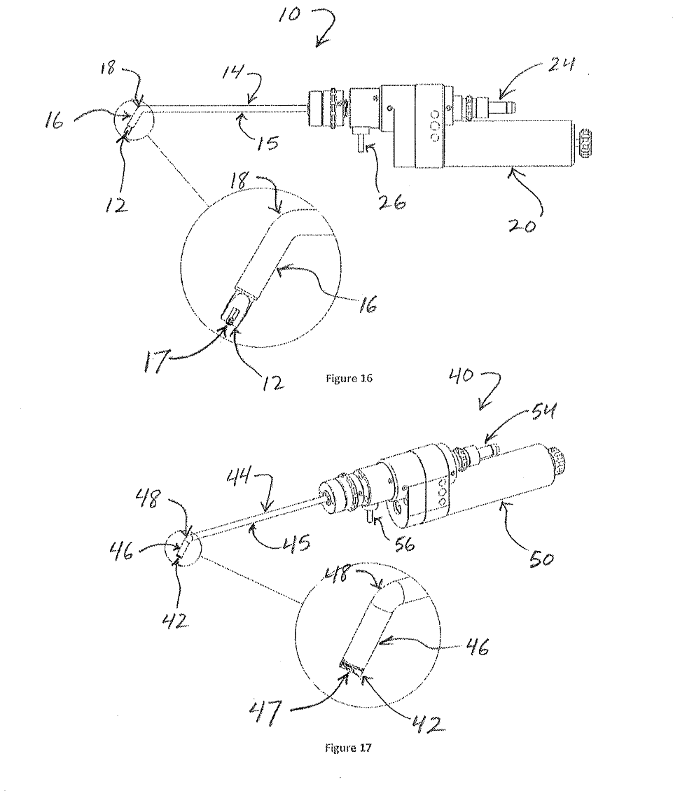

Referring now to FIG. 16, one embodiment of micro-debrider device 10 is shown in more detail. Many of the features and aspects of micro-debrider device 10 are described in greater detail in U.S. patent application Ser. No. 13/007,578 (Pub. No. 2012/0109172), entitled “Selective Tissue Removal Tool for Use in Medical Applications and Methods for Making and Using,” filed on Jan. 14, 2011, which is hereby incorporated by reference in its entirety. As mentioned previously, micro-debrider device 10 may include handle 20 and shaft 14. Shaft 14 may include a proximal portion 15, distal tip 16 and bend 18 at the intersection of proximal portion 15 and distal tip 16. As shown in greater detail in the close-up view, distal tip 16 includes distal end 12 and a cutting member 17 at distal end 12. In this embodiment, cutting member 17 includes multiple, interdigitated blades, which will be described further below.

Handle 20 may include, in some embodiments, a suction port 24 and/or an irrigation port 26 for coupling handle 20 with a source of suction and/or irrigation, respectively. Ports 24, 26 are in fluid communication with one or two channels extending through shaft 14. In some embodiments, for example, shaft 14 may include a suction channel and an irrigation channel. In alternative embodiments, shaft 14 may include one common suction/irrigation channel. In one embodiment with two channels, device 10 may include an inner shaft (not visible in FIG. 16) and an outer shaft 14, with the middle bore of the inner shaft being used as a suction lumen and a space between the outer surface of the inner shaft and the inner surface of outer shaft 14 being used as an irrigation lumen. In an alternative embodiment, the opposite configuration for suction/irrigation may be used.

In general, the outer diameter of shaft 14 may be relatively quite small, since cutting member 17 and the mechanical elements used to drive it are also quite small. This small outer shaft diameter may facilitate use of device 10 within the mouth. The angle of bend 18 and the length of distal tip 16 may also be designed to facilitate usability. In some embodiments, for example, shaft 14 may have an outer diameter of between about 1 mm and about 10 mm, distal tip may have a length of between about 1 mm and about 25 mm, and bend 18 may form an angle of between about 1 degree and about 90 degrees. Even more ideally, in some embodiments, the outer diameter of shaft 14 may be between about 2 mm and about 4 mm.

In various alternative embodiments, bend 18 may be fixed or adjustable. In the embodiments shown and described in FIG. 16 and other figures herein, bend 18 is generally fixed. However, in alternative embodiments, bend 18 may be manually adjustable to adjust the angle or may be mechanically adjustable by the device itself.

Referring now to FIG. 17, an alternative embodiment of micro-debrider device 40 is shown. As with previously described embodiments, micro-debrider device 40 may include a handle 50 and a shaft 44. Shaft 44 may include a proximal portion 45, distal tip 46 and bend 48 at the intersection of proximal portion 45 and distal tip 46. As shown in greater detail in the close-up view, distal tip 46 includes distal end 42 and a cutting member 47 at distal end 42. In this embodiment, cutting member 47 includes a rotating blade that rotates past a stationary blade, as will be described further below. Handle 50 may include, in some embodiments, a suction port 54 and/or an irrigation port 56, for coupling handle 50 with a source of suction and/or irrigation, respectively. Ports 54, 56 are in fluid communication with one or two channels extending through shaft 44.

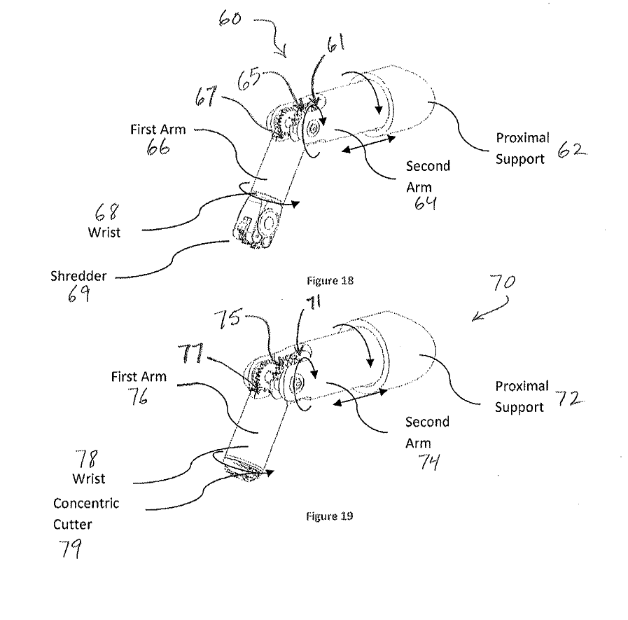

Referring now to FIG. 18, a distal portion of one embodiment of a micro-debrider device 60 is illustrated. In this embodiment, a first arm 66 (or “distal tip”) is connected to a second arm 64 (or “inner shaft”) via a middle gear 65. A proximal support 62 (or “outer shaft”) surrounds at least part of second arm 64. First arm 66 is coupled with a tissue shredder 69 via a wrist 68, which allows shredder 69 to rotate relative to first arm 66. In this embodiment, movement and/or adjustments may occur at wrist 68, and the angle between first arm 66 and second arm 64 is fixed. In various alternative embodiments, first arm 66 and second arm 64 may be adjustable, relative to one another. Such adjustments may be carried out via mechanisms within device 60 or alternatively by adjusting device 60 with the hands or an adjustment tool. In this embodiment, a first crown gear 67 resides inside first arm 66, and a second crown gear 61 resides inside second arm 64. Second crown gear 61 rotates to turn middle gear 65, and middle gear 65 rotates first crown gear 67, which turns the blades of tissue shredder 69. In the embodiment shown, two sets of blades rotate in opposite directions toward one another to cut (or “shred” or “tear”) tissue and also to urge the cut tissue into device 60. The configuration of first crown gear 67, middle gear 65 and second crown gear 61 allows device 60 to have a very sharp bend and a very small diameter, while still providing for effective driving of the blades of tissue shredder 69. Prior art blade driving mechanisms typically do not allow for such sharp bends and/or small diameters, because the mechanisms are not able to drive distal actuated cutters around a tight bend.

Referring now to FIG. 19, a distal portion of another embodiment of a micro-debrider device 70 is illustrated. In this embodiment, a first arm 76 (or “distal tip”) is connected to a second arm 74 (or “inner shaft”) via a middle gear 75. A proximal support 72 (or “outer shaft”) surrounds at least part of second arm 74. First arm 76 is coupled with a concentric cutter 79 via a wrist 68, which allows concentric cutter 79 to rotate relative to first arm 76, while first arm 76 and second arm 74 are fixed relative to one another. As with the previously described embodiment, a first crown gear 77 resides inside first arm 76, and a second crown gear 71 resides inside second arm 74. Second crown gear 71 rotates to turn middle gear 75, and middle gear 75 rotates first crown gear 77, which turns the blade of concentric cutter 79.

Referring now to FIG. 20, an exploded view of a distal portion of one embodiment of a micro-debrider device 80 is shown. FIG. 20 illustrates the gearing mechanism that drives the blade assembly 86 of device 80. Although each part illustrated in FIG. 20 will not be described in detail, the figure should illustrate the parts with sufficient detail to allow one of skill in the art to make and use the gearing. In this embodiment, micro-debrider device 80 includes a housing 88 disposed over a drive tube crown gear 89 (analogous to first gear 67, 77 in FIGS. 18 and 19), which attaches to a lug 90, which holds blade assembly 86. Blade assembly 86 is attached to lug 90 and actuated via two retainers 81, 93, two right angle gears 82, 92, two pins 84, 96, two pin aligners 85, 91, two small gears 83, 95, and two spacers 87, 94. When device 80 is actuated, drive tube crown gear 89 rotates in one direction and then another to drive right angle gears 82, 92 and small gears 83, 95, which in turn drive the blades of blade assembly 86 to rotate in opposite directions relative to one another (i.e., toward one another). As the blades rotate toward one another, they pass very close to one another, thus sheering off tissue (or shredding tissue) between the blades. As described above, the mechanism illustrated in FIG. 20, combined with a middle gear and a second drive tube crown gear, allows device 80 to have a sharp bend and a small outer diameter.

Referring now to FIG. 21, in another embodiment, a micro-debrider device 120 may include an outer tube 122, inner drive tube 124 and other mechanism as described above. This embodiment, however, includes a concentric cutter 126 rather than the reciprocating blades of the embodiment described above. Concentric cutter 126 generally includes a rotating blade and a stationary blade. The rotating blade rotates in one direction passing in close proximity to the stationary blade and thus cutting tissue between the two blades. In some embodiments, the rotating blade and the stationary blade include multiple blades that interdigitate with each other, such that tissue is shredded between the moving and stationary interdigitating blades.

FIG. 22 illustrates an exemplary embodiment of a blade assembly 100 of a micro-debrider tissue removal device. Blade assembly 100, which is similar to those illustrated in lesser detail in FIGS. 16, 18 and 20, is described in further details and in alternative embodiments in U.S. patent application Ser. No. 13/007,578 (Pub. No. 2012/0109172), which was previously incorporated by reference herein. Tissue removal device working end 100 has a distal region “D” and proximal region “P,” and includes housing 101 and blade stacks 102 and 104. Blade stacks 102 and 104 include a plurality of blades 102A-102C and 104A-104C, respectively. Three blades are shown in each stack, although the blade stacks can have one or more blades. Each of the blades includes a plurality of teeth 106, some of which are shown projecting from housing 101 and configured to engage and process tissue. Processing tissue as used herein includes any of cutting tissue, shredding tissue, capturing tissue, any other manipulation of tissue as described herein, or any combination thereof. The working end of the device generally has a length L, height H, and width W. Housing 101 can have a variety of shapes or configurations, including a generally cylindrical shape.

In this embodiment, both blade stacks are configured to rotate. The blades in blade stack 102 are configured to rotate in a direction opposite that of the blades in blade stack 104, as designated by the counterclockwise “CCW” and clockwise “CW” directions in FIG. 22. The oppositely rotating blades direct material, such as tissue, into an interior region of housing 101 (described in more detail below). In some embodiments, the blades can be made to be rotated in directions opposite to those indicated, e.g. to disengage from tissue if a jam occurs or to cause the device to be pulled distally into a body of tissue when given appropriate back side teeth configurations.

Housing 101 also includes a drive mechanism coupler 105, shown as a square hole or bore, which couples a drive train disposed in the housing to a drive mechanism disposed external to the housing. The drive mechanism, described in more detail below, drives the rotation of the drive train, which drives the rotation of the blades. The drive train disposed in the housing can also be considered part of the drive mechanism when viewed from the perspective of the blades. Drive mechanism coupler 105 translates a rotational force applied to the coupler by the drive mechanism (not shown) to the drive train disposed within housing 101. FIG. 22 also shows release holes 111-115 which allow for removal of sacrificed material during formation of the working end.

Material may be directed into housing 101 by the rotating blades, and housing may include a chamber (not visible) where the cut tissue can be stored temporarily or directed further proximally. In some embodiments in which the working end 100 includes a storage chamber, the chamber may remain open while in other embodiments it may be closed while in still other embodiments it may include a filter that only allows passage of items of a sufficiently small size to exit.

In general, the blades in stack 102 are interdigitated with the blades in stack 104 (i.e. the blade ends are offset vertically along dimension H and have maximum radial extensions that overlap laterally along the width dimension W. The blades can be formed to be interdigitated by, e.g. if formed using a multi-layer, multi-material electrochemical fabrication technique, forming each blade in stack 102 in a different layer than each blade in stack 104. If during formation portions of separately moveable blade components overlap laterally, the overlapping blades should not just be formed on different layers but should be formed such an intermediate layer defines a vertical gap between them. For example, the bottom blade in stack 102 is shown formed in a layer beneath the layer in which the bottom blade in stack 104 is formed.

When manufacturing tissue removal devices of the various embodiments set forth herein using a multi-layer multi-material electrochemical fabrication process, it is generally beneficial, though not necessarily required, to maintain horizontal spacing of component features and widths of component dimensions remain above the minimum feature size. It is important that vertical gaps of appropriate size be formed between separately movable components that overlap in X-Y space (assuming the layers during formation are being stacked along the Z axis) so that they do not inadvertently bond together and to ensure that adequate pathways are provided to allow etching of sacrificial material to occur. For example, it is generally important that gaps exist between a gear element (e.g. a tooth) in a first gear tier and a second gear tier so that the overlapping teeth of adjacent gears do not bond together. It is also generally important to form gaps between components that move relative to one another (e.g., gears and gear covers, between blades and housing, etc.). In some embodiments the gaps formed between moving layers is between about 2 um and about 8 um.

In some embodiments, it is desired to define a shearing thickness as the gap between elements has they move past one another. Such gaps may be defined by layer thickness increments or multiples of such increments or by the intralayer spacing of elements as they move past one another. In some embodiments, shearing thickness of blades passing blades or blades moving past interdigitated fingers, or the like may be optimally set in the range of 2-100 microns or some other amount depending on the viscosity or other parameters of the materials being encountered and what the interaction is to be (e.g. tearing, shredding, transporting, or the like). For example, for shredding or tearing tissue, the gap may be in the range of 2-10 microns, or in some embodiments in the range of 4-6 microns.

Referring now to FIG. 23, in one alternative embodiment, a blade assembly 130 may include a concentric cutter. In this embodiment, blade assembly 130 includes a rotating (or “concentric”) cutter 132 having multiple blades 132a, 132b, 132c and a stationary cutter 134 having multiple blades 134a, 134b, 134c. Rotating blades 132a, 132b, 132c interdigitate with stationary blades 134a, 134b, 134c so that tissue is cut off between them. As with the previously described embodiment, tissue that is cut or shredded by cutters 132, 134 is typically urged proximally into blade assembly 130 and thus into a chamber and/or conduit of the device. In some embodiments, this proximal movement may be facilitated by suction and/or irrigation.

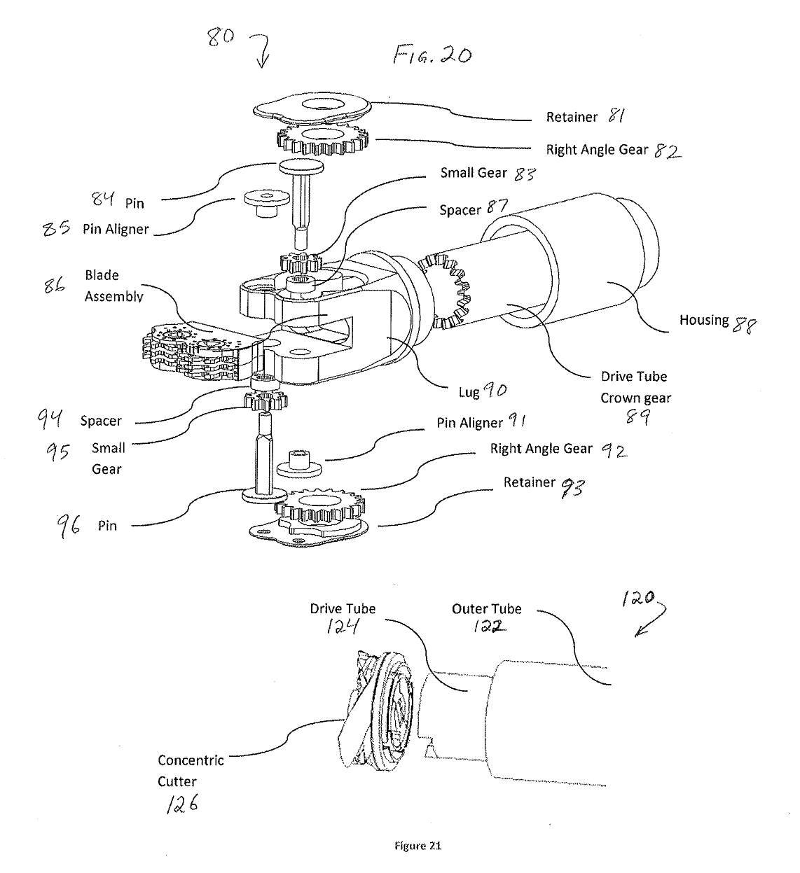

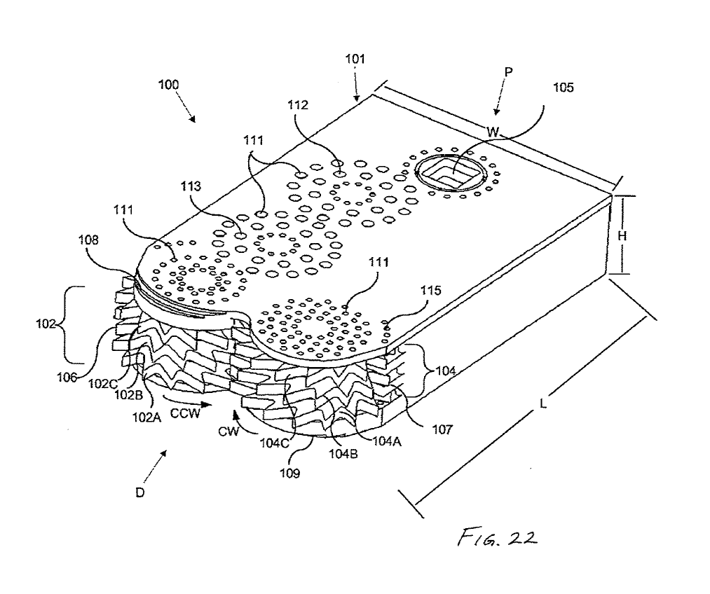

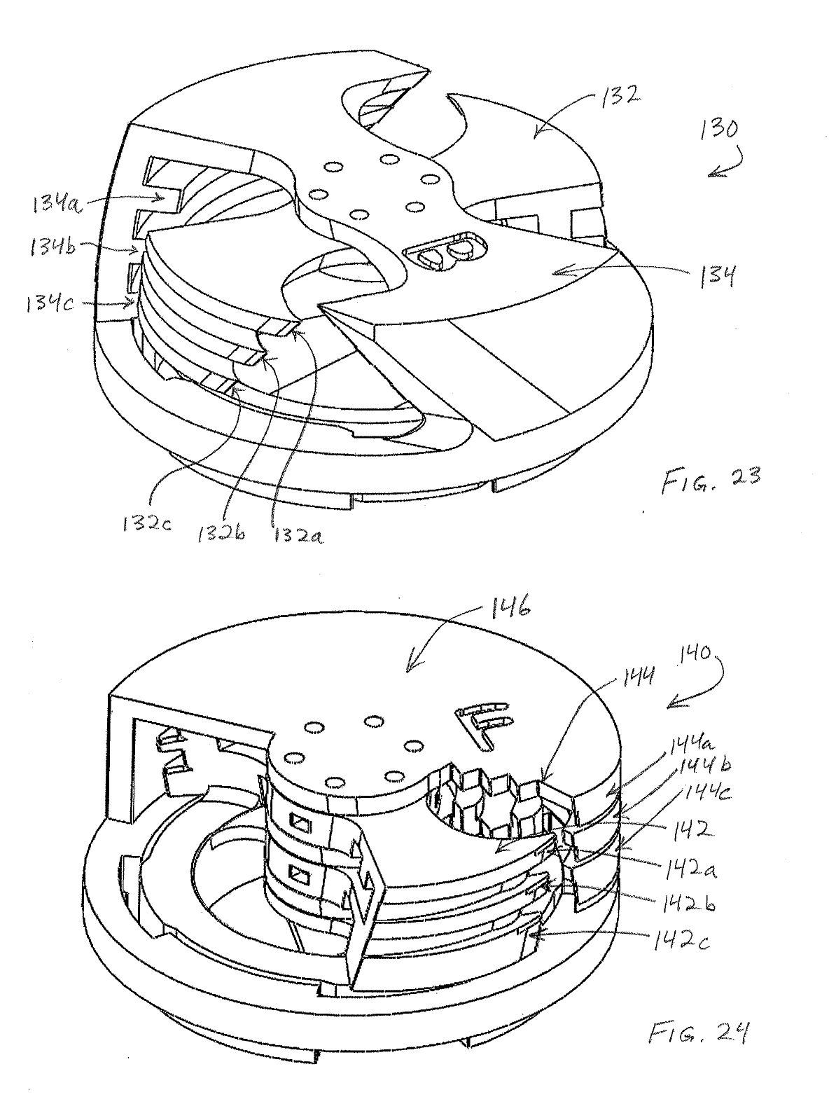

With reference now to FIG. 24, in another alternative embodiment, a blade assembly 140 may include a concentric cutter. In this embodiment, blade assembly 140 includes a rotating (or “concentric”) cutter 142 having multiple blades 142a, 142b, 142c and a stationary cutter 144 having multiple blades 144a, 144b, 144c. Rotating blades 142a, 142b, 142c interdigitate with stationary blades 144a, 144b, 144c so that tissue is cut off between them. As with the previously described embodiment, tissue that is cut or shredded by cutters 142, 144 is typically urged proximally into blade assembly 140 and thus into a chamber and/or conduit of the device. This embodiment also includes a guard portion 146 on top (i.e., the extreme distal end) of blade assembly 140. Guard portion 146 may help protect nearby tissues from unwanted damage and may thus help facilitate tissue removal procedures near sensitive structures.

Elements or components shown with any embodiment herein are exemplary for the specific embodiment and may be used on or in combination with other embodiments disclosed herein. The invention is susceptible to various modifications and alternative forms and should not be limited to the particular forms or methods disclosed. To the contrary, the invention is to cover all modifications, equivalents and alternatives thereof.

1. A method for removing a volume of tissue from a tongue in a patient to treat sleep apnea, the method comprising:
cutting tissue from the tongue using a tissue cutting device having a shaft and at least one moveable cutting member attached to the shaft at a distal end of the tissue cutting device; and
moving the cut tissue through a channel of the shaft in a direction from the distal end of the tissue cutting device toward a proximal end of the device.

2. A method as in claim 1, further comprising, before cutting the tissue:
forming an incision in the tongue; and
advancing the distal end of the tissue cutting device through the incision to cut tissue within an inner portion of the tongue.

3. A method as in claim 2, wherein the incision is formed using the tissue cutting device.

4. A method as in claim 2, wherein the incision is formed in a top of the tongue.

5. A method as in claim 2, wherein the incision is formed in a bottom of the tongue.

6. A method as in claim 2, wherein the incision is formed from under the patient's chin through a bottom of the tongue.

7. A method as in claim 2, further comprising closing the incision using an energy emitting member on the tissue cutting device, wherein the energy emitting member emits energy selected from the group consisting of radiofrequency, ultrasound, microwave, heat and laser energy.

8. A method as in claim 1, wherein the moveable cutting member comprises at least one moveable blade and at least one stationary blade, and wherein cutting tissue comprises rotating the at least one rotating blade past the at least one stationary blade.

9. A method as in claim 1, wherein the moveable cutting member comprises at least two interdigitated tissue cutters, and wherein cutting tissue comprises rotating the two interdigitated cutters toward one another.

10. A method as in claim 1, wherein moving the cut tissue through the channel comprises applying suction to the channel.

11. A method as in claim 10, wherein moving the cut tissue through the channel further comprises introducing fluid, via the tissue cutting device, to an area at or near the distal end of the tissue cutting device, wherein the applied suction moves at least some of the fluid proximally through the channel with the cut tissue.

12. A method as in claim 1, wherein the shaft of the tissue cutting device has a diameter no greater than about 10 mm, a distal tip having a length of between about 1 mm and about 25 mm, and a bend between a proximal portion of the shaft and the distal tip forming an angle between the proximal portion and the distal tip of between about 1 degree and about 90 degrees.

13. A method as in claim 1, further comprising visualizing the cutting using a visualization device selected from the group consisting of a straight endoscope, an angled endoscope, a swing prism endoscope, a side viewing endoscope, a flexible endoscope, a CMOS digital camera, an ultrasound device and a scanning single fiber endoscope.

14. A method as in claim 13, wherein the visualization device is incorporated into the tissue removal device.

15. A method as in claim 1, further comprising measuring an amount of the removed tissue by filtering the removed tissue from a stream of irrigation fluid.

16. A method as in claim 1, further comprising measuring an amount of the removed tissue by determining motor torque in the tissue removal device during engagement of the device with the tissue and using at least one of the determined motor torque, a time period of tissue removal or a loading condition to approximate the amount of the removed tissue.

17. A method for removing a volume of tissue from a tongue in a patient to treat sleep apnea, the method comprising cutting tissue from the tongue using a mechanical, tissue debriding device comprising at least one moveable blade.

18. A device for removing a volume of tissue from a tongue in a patient to treat sleep apnea, the device comprising:
a shaft having a proximal portion, a distal tip disposed at an angle relative to the proximal portion, and a channel extending from a distal end of the distal tip through at least part of the proximal portion;
at least one moveable cutting member disposed at the distal end of the distal tip and including at least two interdigated blades;
a handle coupled with the proximal portion of the shaft; and
an actuator coupled with the handle for actuating the at least one moveable cutting member.

19. A device as in claim 18, wherein the shaft has a diameter no greater than about 10 mm, a distal tip having a length of between about 1 mm and about 25 mm, and a bend between a proximal portion of the shaft and the distal tip forming an angle between the proximal portion and the distal tip of between about 1 degree and about 90 degrees.

20. A device as in claim 18, wherein the channel comprises a tissue removal channel extending from the distal end of the distal tip to a proximal aperture on the proximal portion through which tissue can be removed from the device.

21. A device as in claim 18, further comprising a suction port on the proximal portion or the handle for applying suction to the channel.

22. A device as in claim 21, further comprising an irrigation port on the proximal portion or the handle for applying irrigation fluid to the channel.

23. A device as in claim 22, wherein the suction port is in fluid communication with a suction channel in an inner tube of the device, and wherein the irrigation port is in fluid communication with an irrigation channel comprising a space between an outer surface of the inner tube and an inner surface of the shaft of the device.

24. A device as in claim 18, wherein the at least one moveable cutting member comprises:
at least one rotating blade; and
at least one stationary blade positioned relative to the rotating blade such that tissue is cut between the rotating blade and the stationary blade.

25. A device as in claim 18, wherein the at least one moveable cutting member comprises multiple interdigitated cutters that rotate toward one another to shred tissue.

26. A device as in claim 18, further comprising at least one tubular crown gear for driving the at least one cutting member.

27. A device as in claim 26, wherein the at least one tubular crown gear comprises two tubular crown gears coupled together with at least one intermediate gear disposed between them.

28. A device as in claim 27, wherein the intermediate gear is disposed at a bend in the shaft located at an intersection of the proximal portion and the distal tip.

29. A device as in claim 18, further comprising an energy transmission member coupled with the distal tip of the shaft for transmitting energy to the tissue, wherein the energy transmitted by the energy transmission member is selected from the group consisting of radiofrequency, ultrasound, microwave, heat and laser energy.

30. A system for removing a volume of tissue from a tongue in a patient to treat sleep apnea, the system comprising:
a mechanical tissue debrider, comprising:
a shaft having a proximal portion, a distal tip disposed at an angle relative to the proximal portion, and a channel extending from a distal end of the distal tip through at least part of the proximal portion;
at least one moveable cutting member disposed at the distal end of the distal tip;
a handle coupled with the proximal portion of the shaft; and
an actuator coupled with the handle for actuating the at least one moveable cutting member;
an energy transmission member coupled with the distal tip of the shaft for transmitting an energy to the tissue, wherein the energy is selected from the group consisting of radiofrequency, ultrasound, microwave, heat and laser energy.

31. A system as in claim 30, further comprising a suction port on the proximal portion of the shaft or the handle for applying suction to the channel.

32. A device as in claim 31, further comprising an irrigation port on the proximal portion of the shaft or the handle for applying irrigation fluid to the channel.
